(19) United States
(12) Patent Application Publication
(10) Pub. No.: US 2005/0130159 A1
(43) Pub. Date: Jun. 16, 2005

(54) Sequencing on perforated membranes

(75) Inventors: Rudolf Rigler, St-Sulpice (CH); Horst Vogel, Preverenges (CH)

Correspondence Address: THE WEBB LAW FIRM, P.C., 700 KOPPERS BUILDING, 436 SEVENTH AVENUE, PITTSBURGH, PA, 15219, US

(73) Assignee: GNOTHIS HOLDING SA, Ecublens (CH)

(21) Appl. No.: 10/503,562

(22) PCT Filed: Dec. 18, 2002
(86) PCT No.: PCT/EP02/14489

(30) Foreign Application Priority Data
Dec. 19, 2001 (DE) 101 62 535.9

(51) Int. Cl. C12Q 1/68; C07H 21/04; C12M 1/34

(52) U.S. Cl. 435/6; 435/287.2; 536/25.32

(57) ABSTRACT

The invention concerns a method and device for sequencing nucleic acid molecules on a perforated membrane. The invention can be used in particular for multiplex sequencing.

The invention concerns a method and device for sequencing nucleic acid molecules on a perforated membrane. The invention can be used in particular for multiplex sequencing.

The sequencing of the human-genome composed of about 3×10⁹ bases or of the genome of other organisms as well as the determination and comparison of individual sequence variants requires the provision of sequencing methods which are, on the one hand, rapid and which on the other hand, can be used routinely and at low costs. Major efforts have been made in recent years to accelerate the current sequencing methods e.g. the enzymatic chain termination method according to Sanger et al. (Proc. Natl. Acad. Sci. USA 74 (1977) 5463), in particular by automation (Adams et al., Automated DNA Sequencing and Analysis (1994), New York, Academic Press). At present a maximum of up to 500 000 bases can be determined per day with a sequencer. Nevertheless, conventional sequencing methods are unsuitable or of only limited suitability for some applications.

New approaches for overcoming the limitations of conventional sequencing methods have been developed in recent years inter alia sequencing by scanning-tunnel microscopy (Lindsay and Phillip, Gen. Anal. Tech. Appl. 8 (1991), 8-13), by highly parallelized capillary electrophoresis (Huang et al., Anal. Chem. 64 (1992), 2149-2154; Kambara and Takahashi, Nature 361 (1993), 565-566), by oligonucleotide hybridization (Drmanac et-al., Genomics 4 (1989), 114-128; Khrapko et al., FEBS Let. 256 (1989), 118-122; Maskos and Southern, Nucleic Acids Res. 20 (1992), 1675-1678 and 1679-1684) and by matrix-assisted laser desorption/ionization mass spectrometry (Hillenkamp et al., Anal. Chem. 63 (1991), 1193A-1203A).

Another approach is single molecule sequencing (Dörre et al., Bioimaging 5 (1997), 139-152) in which the sequence of nucleic acids is determined by successive enzymatic degradation of fluorescent-labelled single-stranded DNA molecules and detection of the sequentially released monomer molecules in a microstructured channel. The advantage of this method is that only a single molecule of the target nucleic acid is sufficient to carry out a sequence determination.

Although considerable improvements have been achieved by using the above-mentioned methods there is a major need for further improvements. Hence the object of the present invention was to provide a method for sequencing nucleic acids which is a further improvement over the prior art and which enables a parallel determination of single nucleic acid molecules in a multiplex format.

This object is achieved by a method for sequencing nucleic acids comprising the steps:
(a) providing a nucleic acid molecule to be sequenced which contains a plurality of fluorescence-labelling groups,
(b) providing a membrane structure through which at least one channel extends which has a suitable diameter for the passage of single nucleic acid molecules, wherein an enzyme which catalyses the cleavage of single nucleotide building blocks from a nucleic acid molecule is immobilized on the membrane structure,
(c) passing the nucleic acid molecule to be sequenced into and/or through the channel under conditions that allow a successive cleavage of single nucleotide building blocks from the nucleic acid molecule and
(d) determining the base sequence of the nucleic acid molecule by fluorescence measurement.

The method according to the invention is a carrier-based sequencing method in which a free nucleic acid molecule to be sequenced is passed into and preferably through a channel in a membrane structure and is brought into contact with an enzyme during passage through the channel and/or preferably when it passes out of the channel, said enzyme catalysing the cleavage of single nucleotide building blocks from the nucleic acid molecule. The enzyme is immobilized on the membrane structure preferably in the area of the outlet ports of the channel. The membrane structure preferable contains a plurality of channels and can thus be used to simultaneously determine the base sequence of a plurality of nucleic acid molecules.

The membrane structure can have any shape and composition provided it is suitable for immobilizing enzymes and for forming nanochannels for passage of the nucleic acid molecules to be sequenced. Examples of suitable materials are glass, plastic, metals or semimetals such as silicon, metal oxides such as silicon oxide, quartz etc. Moreover, composite materials that are for example made of two or more of the aforementioned materials are also suitable.

The enzyme molecules are immobilized on the membrane structure in particular in the area of the outlet ports of the channels by means of known methods. The enzyme molecules can bind to the membrane by means of covalent or non-covalent interactions. For example the binding of the enzyme molecules to the membrane structure can be mediated by high-affinity interactions between partners of a specific binding pair e.g. biotin/streptavidin or avidin, hapten/anti-hapten antibody, sugar/lectin etc. Thus biotinylated enzyme molecules can be coupled to streptavidin-coated membrane structures. Alternatively the enzyme molecules can also be bound adsorptively to the membrane structure. Thus enzyme molecules modified by incorporation of alkanethiol groups can be bound to metallic carriers e.g. gold carriers. Still another alternative is covalent immobilization in which the binding of the enzyme molecules can be mediated by suitable (hetero)bifunctional coupling reagents.

The method according to the invention is preferably carried out as a multiplex method for sequencing a plurality of nucleic acid molecules. For this it is advantageous to use a membrane structure that contains a plurality of channels. The average diameter of the channels is preferably in the range of 10-100 nm in order to enable the passage in each case of single nucleic acid molecules to be sequenced. Preferably at least 10, more preferably at least 20, particularly preferably at least 100 and most preferably 1000 or more nucleic acid molecules are sequenced in parallel.

The nucleic acid molecules to be sequenced have a length of preferably at least 100 nucleotides, particularly preferably of at least 200 nucleotides. Basically the nucleic acid molecules can be of any length e.g. several kb or even longer. The maximum length is only determined by the lifetime of the immobilized enzyme. The nucleic acid molecules e.g. DNA molecules or RNA molecules contain a plurality of fluorescence-labelling groups, wherein preferably at least 50%, particularly preferably at least 70% and most preferably essentially all e.g. at least 90% of the nucleotide building blocks of one base type carry a fluorescence-labelling group. Such labelled nucleic acids can be produced by enzymatic primer extension on a nucleic acid template using a suitable polymerase e.g. a DNA polymerase such as Taq polymerase, a thermostable DNA polymerase from Thermococcus gorgonarius or other thermostable organisms (Hopfner et al., PNAS USA 96 (1999), 3600-3605) or a mutated Taq polymerase (Patel and Loeb, PNAS USA 97 (2000), 5095-5100) using fluorescent-labelled nucleotide building blocks.

The labelled nucleic acid molecules can also be produced by amplification reactions e.g. PCR. Thus in an asymmetric PCR, amplification products are formed where only a single strand contains fluorescent labels. Such asymmetric amplification products can be sequenced in a double-stranded form. Nucleic acid fragments are produced by symmetric PCR where both strands are fluorescent-labelled. These two fluorescent-labelled strands can be separated and introduced separately in a sequencing device such that the sequence of one or both complementary strands can be determined separately. Alternatively one of the two strands can be modified at the 3′ end e.g. by incorporation of a PNA clamp such that monomer building blocks can no longer be cleaved. In this case double-stranded sequencing is possible.

Preferably essentially all nucleotide building blocks of at least two base types, for example two, three or four base types, carry a fluorescence label where each base type advantageously carries a different fluorescence-labelling group. If the nucleic acid molecules are not completely labelled, it is nevertheless possible to determine the complete sequence by sequencing several molecules in parallel.

The nucleic acid template whose sequence is to be determined can for example be selected from DNA templates such as genomic DNA fragments, cDNA molecules, plasmids etc. but also from RNA templates such as mRNA molecules.

The fluorescence-labelling groups can be selected from known fluorescence-labelling groups for labelling biopolymers, e.g. nucleic acids, such as fluorescein, rhodamine, phycoerythrin, Cy3, Cy5 or derivatives thereof etc.

The method according to the invention is preferably based on the fact that fluorescence-labelling groups incorporated into nucleic acid strands interact with neighbouring groups, for example with chemical groups of nucleic acids, in particular nucleobases such as G, or/and with neighbouring fluorescence-labelling groups which results in a change in the fluorescence and in particular of the fluorescence intensity compared to the fluorescence-labelling groups in an isolated form due to quenching or/and energy transfer processes. Cleavage of single nucleotide building blocks changes the total fluorescence e.g. the fluorescence intensity of a nucleic acid strand dependent on the cleavage of single nucleotide building blocks i.e. in a time-dependent manner. This time-dependent change in fluorescence can be determined concurrently for a plurality of nucleic acid molecules and be correlated with the base sequence of the individual nucleic acid strands. It is preferable to use fluorescence-labelling groups which are at least partially quenched when they are incorporated into the nucleic acid strand so that the fluorescence intensity is increased after cleavage of the nucleotide building block containing the labelling group or of a neighbouring building block which causes quenching.

The sequencing reaction of the method according to the invention comprises the successive cleavage by immobilized enzymes of individual nucleotide building blocks from the nucleic acid molecules passed through the channel. They are preferably cleaved enzymatically using an exonuclease in which case single strand or double strand exonucleases that degrade in the 5′→3′ direction or 3′→5′ direction can be used depending on the type of immobilization of the nucleic acid strands on the carrier. T7 DNA polymerase, E.coli exonuclease I or E.coli exonuclease III are particularly preferably used as exonucleases.

During the successive cleavage of single nucleotide building blocks it is possible to measure a change in the fluorescence intensity of the immobilized nucleic acid strands or/and of the cleaved nucleotide building block due to quenching or energy transfer processes. This time-dependent change in fluorescence intensity is dependent on the base sequence of the examined nucleic acid strand and can therefore be correlated with the sequence. In order to determine the complete sequence of a nucleic acid strand, a plurality of nucleic acid strands labelled on different bases e.g. A, G, C and T and/or combinations of two different bases are preferably generated by enzymatic primer extension as previously described and passed successively through a channel or/and through different channels of the membrane structure. Where necessary, a sequence identifier i.e. a labelled nucleic acid of known sequence can be attached to the nucleic acid strand to be examined e.g. by enzymatic reaction with ligase or/and terminal transferase such that at the start of sequencing a known fluorescence pattern is firstly obtained and only subsequently the fluorescence pattern corresponding to the unknown sequence to be examined. A total of preferably at least 10 and up to more than 1000 nucleic acid strands can be sequenced in parallel on a carrier.

The nucleic acid molecules to be sequenced can for example be passed through the channels of the membrane structure by a hydrodynamic or/and electroosmotic flow. The passage of the nucleic acid molecules to be sequenced particularly preferably comprises applying an electric field across the membrane which results in a migration from − to + due to the negative charge of the nucleic acid molecules under physiological conditions.

The detection preferably comprises a multipoint fluorescence excitation by a laser e.g. a point matrix of laser points generated by diffraction optics or a quantum well laser. The fluorescence emission of a plurality of nucleic acid strands generated by the excitation can be detected by a detector matrix which for example comprises an electronic detector matrix e.g. a CCD camera or an avalanche photodiode matrix. The detection can be such that the fluorescence excitation and detection of all examined nucleic acid strands is carried out in parallel. Alternatively a portion of the nucleic acid strands can be examined in each case in several steps using a submatrix of laser points and detectors and preferably using a high-speed scanner procedure.

Another subject matter of the invention is a carrier for sequencing nucleic acids comprising a membrane structure through which at least one channel extends, an enzyme which catalyses the cleavage of single nucleotide building blocks from a nucleic acid molecule being immobilized on the membrane structure in the area of the channel or the channels. The diameter of the channel is such that single nucleic acid molecules can pass. The diameter is preferably between 10 and 100 nm. The membrane structure preferably contains a plurality of channels for the concurrent sequencing of a plurality of identical or/and different nucleic acid molecules.

Another subject matter of the invention is a device for sequencing nucleic acids comprising:
(a) a carrier as specified above,
(b) means for passing the nucleic acid molecules to be sequenced through the channels of the carrier and
(c) means for simultaneously determining the base sequence of a plurality of nucleic acid molecules on the basis of the time-dependent change in the fluorescence of the nucleic acid molecules or/and of the cleaved nucleotide building blocks caused by the cleavage of nucleotide building blocks.

The method according to the invention can for example be used to analyse genomes and transcriptomes and/or for differential analyses e.g. to investigate differences in the genome and/or transcriptome of individual species or organisms within a species.

The present invention is further elucidated by the following figures.

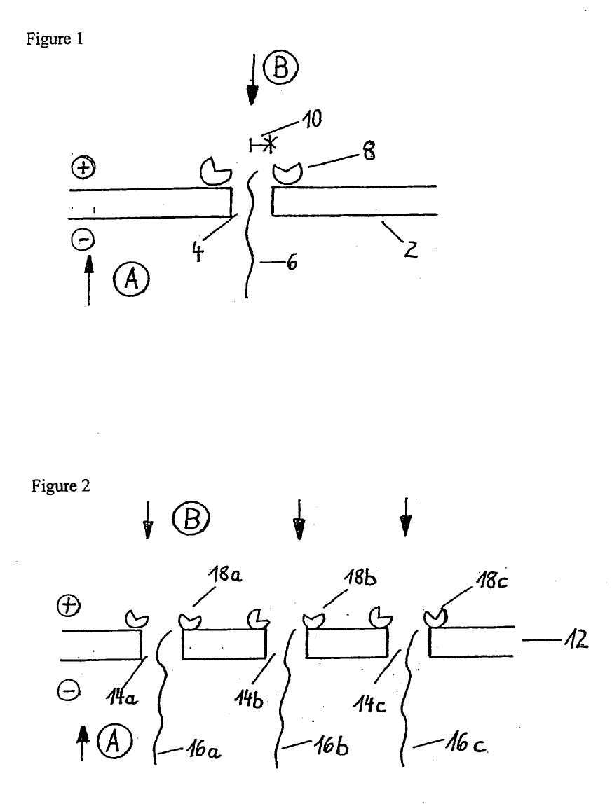

FIG. 1 shows a schematic representation of an embodiment of the method according to the invention. A membrane structure (2) contains a nanochannel (4) for the passage of single nucleic acid molecules to be sequenced (6). The migration direction of the nucleic acid molecules through the channel is indicated by an arrow (A). An electric field can be applied across the membrane to facilitate the migration. As the nucleic acid molecules to be sequenced (6) pass through the channel (4) they come into contact with exonuclease molecules (8) immobilized on the membrane structure (2). The exonuclease molecules (8) are located especially in the area of the channel ports. The exonuclease molecules (8) cleave the nucleic acid molecules (6) passing through the channel (4) to form cleavage fragments (e.g. 10). The fluorescence of the nucleic acid molecules (6) or/and of the cleavage fragments (10) is detected by fluorescence excitation (e.g. as shown by the arrow B) and measurement of the emitted fluorescent light.

FIG. 2 shows a preferred embodiment of the method according to the invention for multiplex sequencing. The membrane structure (12) contains a plurality of channels (14a, 14b, 14c) which can be used to concurrently sequence identical or different nucleic acid molecules (16a, 16b, 16c). Exonuclease molecules (18a, 18b, 18c) are immobilized at each of the channel ports. The sequencing can be carried out using a light source matrix e.g. several laser beams or a laser beam split by a diffraction element or/and a detector matrix.

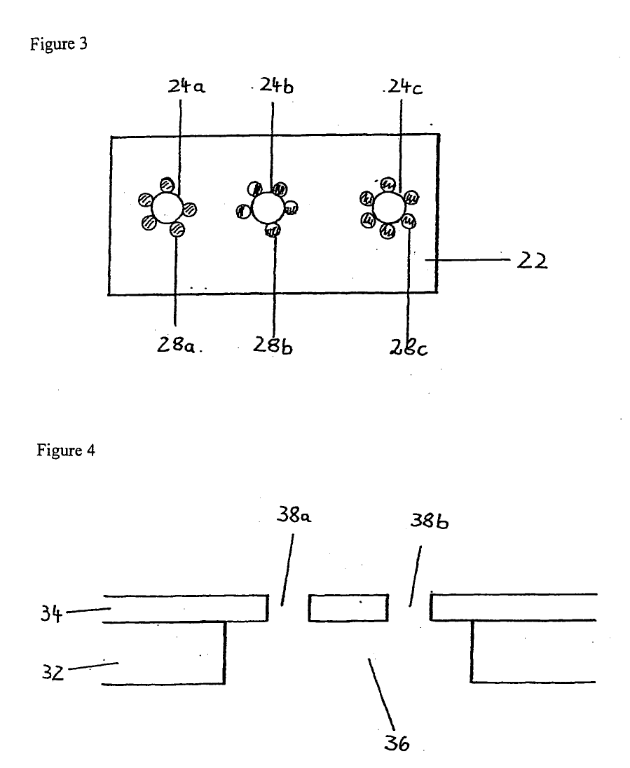

FIG. 3 shows a top-view of a membrane structure (22) containing several channels (24a, 24b, 24c) which is suitable for multiplex sequencing. Exonuclease molecules (28a, 28b, 28c) are immobilized on the membrane structure (22) at least in the area of the channel ports. During passage of the nucleic acid molecules to be sequenced (not shown) through the channel ports fluorescentce-labelled nucleotide building blocks are cleaved off. The release of the nucleotide building blocks increases the fluorescence intensity due to the decrease in quenching resulting in a characteristic photon flow for each base which is characterized by a certain wavelength λ or/and a lifetime τ and can be detected by a detector matrix.

A multilayer carrier is used in the embodiment of the method according to the invention shown in FIG. 4. This carrier comprises a membrane structure composed of several layers (32, 34) comprising a first layer (32) containing a first channel (36) which has a diameter that is sufficient to allow the passage of a plurality of nucleic acids e.g. of 500 to 2000 in particular ca. 1000 nm. After passage through the first channel (36) the nucleic acid molecules to be sequenced arrive at the second layer (34) which is in the form of a cover membrane over the first layer and has one or more second channels (38a, 38b) which have a diameter of e.g. 10 to 100 nm which is suitable for the passage of single nucleic acid molecules. A plurality of identical or different nucleic acid molecules to be sequenced can be passed simultaneously through the first channel (36) to the second layer (34) of the membrane structure and passed individually into the second channels (38a, 38b) and then be sequenced by cleavage of single nucleotide building blocks as elucidated above.

In the case of a membrane structure consisting of several layers, the, sequencing can also be carried out by means of an evanescence-based method. The excitation light originating from an excitation light source e.g. a laser is beamed into an optically transparent layer of the membrane which serves as a carrier of the evanescent wave. Photons are scattered out in the area of the channels which can then excite the cleavage products that are formed there to fluoresce. The fluorescence emission light that is irradiated essentially perpendicularly from the carrier is detected.

1-26. (canceled)

27. A method for sequencing nucleic acids comprising the steps:
(a) providing a nucleic acid molecule to be sequenced which contains a plurality of flourescence-labelling groups,
(b) proving a membrane structure through which at least one channel extends having a suitable diameter for the passage of single nucleic acid molecules, wherein an enzyme which catalyses the cleavage of single nucleotide building blocks from a nucleic acid molecule is immobilized on the membrane structure,
(c) passing the nucleic acid molecule to be sequenced into and/or through the channel under conditions that allow successive cleavage of single nucleotide building blocks from the nucleic acid molecule and
(d) determining the base sequence of the nucleic acid molecule by fluorescence measurement.

28. The method as claimed in claim 27, wherein the nucleic acid molecule to be sequenced is present in a single-stranded form.

29. The method as claimed in claim 27, wherein the nucleic acid molecule to be sequenced is present in a double-stranded form wherein labelled nucleotide building blocks can only be cleaved from a single strand.

30. The method as claimed in claim 27, wherein the nucleic molecules are labelled in such a manner that at least 50% of all nucleotide building blocks of one base type carry a fluorescence-labelling group.

31. The method as claimed in claim 30, wherein essentially all nucleotide building blocks of one base type carry a fluorescence-labelling group.

32. The method as claimed in claim 27, wherein the membrane structure comprises one or more of metals, semimetals, metal oxides, glass, quartz, plastics and composite materials.

33. The method as claimed in claim 27, wherein the channel has a diameter of 10-100 nm.

34. The method as claimed in claim 27, wherein the membrane structure comprises a first layer having a first channel which has a diameter that is adequate for the passage of several nucleic acid molecules, and a second layer having a second channel which has a suitable diameter for the passage of single nucleic acid molecules.

35. The method as claimed in claim 27, wherein the enzyme comprises an exonuclease.

36. The method as claimed in claim 34, wherein the enzyme is selected from the group consisting of T7 DNA polymerase, E. coli exonuclease I and E. coli exonuclease III.

37. The method as claimed in claim 27, wherein step (c) comprises a hydrodynamic or/and an electroosmotic flow through the channel.

38. The method as claimed in claim 27, wherein step (c) comprises applying an electric field across the membrane.

39. The method as claimed in claim 27, wherein the enzyme is immobilized on the membrane structure in the area of the outlet port of the channel.

40. The method as claimed in claim 27, wherein the base sequence is determined on the basis of the time-dependent change in the fluorescence of the nucleic acid molecule or/and of the cleaved nucleotide building blocks caused by the cleavage of nucleotide building blocks.

41. The method as claimed in claim 27, wherein a plurality of nucleic acid molecules to be sequenced are passed into respective separate channels of the membrane structure and the base sequence of this plurality of nucleic acid molecules is determined simultaneously.

42. The method as claimed in claim 41, wherein the simultaneous determination of the base sequence of a plurality of nucleic acid molecules comprises a multipoint fluorescence excitation by a laser.

43. The method as claimed in claim 41, wherein the simultaneous determination of the base sequence of a plurality of nucleic acid molecules comprises a detection of the fluorescence emission of a plurality of nucleic acid strands by a detection matrix.

44. The method as claimed in claim 43, wherein a CCD camera or an avalanche photodiode matrix is used.

45. The method as claimed in claim 41, wherein fluorescence excitation and detection is carried out in parallel on all examined nucleic acid strands.

46. The method as claimed in claim 41, wherein fluorescence excitation and detection are carried out in several steps in each case on a portion of the examined nucleic acid strands using a submatrix of laser points and detectors.

47. The method as claimed in claim 27, wherein the fluorescence-labelling groups are at least partially quenched when they are incorporated into the nucleic acid strands and the fluorescence intensity is increased after cleavage.

48. A carrier for sequencing nucleic acids, comprising: a membrane structure through which a channel extends which has a diameter suitable for the passage of single nucleic acid molecules, an enzyme which catalyses cleavage of single nucleotide building blocks from a nucleic acid molecule being immobilized on the membrane structure.

49. The carrier as claimed in claim 48, wherein the channel has a diameter of 10-100 nm.

50. The carrier as claimed in claim 48, wherein the membrane structure has a plurality of channels for the concurrent sequencing of a plurality of nucleic acids.

51. A device for sequencing nucleic acids comprising:
(a) a carrier as claimed in claim 48,
(b) means for passing the nucleic acid molecules to be sequenced through the channels of the carrier and
(c) means for simultaneously determining the base sequence of a plurality of nucleic acid molecules on the basis of the time-dependent change in the fluorescence of the nucleic acid molecules or/and of the cleaved nucleotide building blocks caused by the cleavage of nucleotide building blocks.
